(19) United States
(12) Patent Application Publication
(10) Pub. No.: US 2007/0036846 A1
(43) Pub. Date: Feb. 15, 2007

(54) Wound dressing and method of manufacture thereof

(76) Inventor: Wing Wai Michael Tsang, New Territories (HK)

Correspondence Address: SMITH-HILL AND BEDELL, P.C., 16100 NW CORNELL ROAD, SUITE 220, BEAVERTON, OR, 97006, US

(21) Appl. No.: 11/204,499

(22) Filed: Aug. 15, 2005

(51) Int. Cl. A61K 31/205; A61L 15/00

(52) U.S. Cl. 424/445; 514/554

(57) ABSTRACT

A hydrogel dressing for covering or treating a wound, a surgical incision, a catheter insertion site or the like, comprising a non-fluid sheet material including a structure of cross-linked polymers and/or monomers having suspended therein an agent for controlling infection of the wound, the incision, the catheter insertion site or the like.

### FIELD OF THE INVENTION

The present invention is concerned with a dressing for covering or treating a wound, a surgical incision, a catheter insertion site or the like.

### BACKGROUND OF THE INVENTION

In the field of wound treatment, there are a variety of products ranging from topical medicated solutions to conventional gauze dressings. Conventional gauze dressings while are useful in treating many types of wounds do not provide any moisture that some wounds may need, and they also do not provide any or sufficient disinfecting effect to the wounds which would often become infected easily. U.S. Pat. No. 4,871,490 discloses the use of a hydrogel dressing. This dressing is useful in keeping the wound moist but it still does not solve the problem of wound infection.

The present invention seeks to provide a wound dressing which possess most if not all the advantages of conventional dressings, overcome the problem of wound infection or the like, or at least to provide a useful alternative to the public.

### SUMMARY OF THE PRESENT INVENTION

According to a first aspect of the present invention, there is provided a dressing for covering or treating a wound, a surgical incision, a catheter insertion site or the like, comprising a non-fluid sheet material including a structure of cross-linked polymers and/or monomers having suspended therein an agent for controlling infection of the wound, the incision, the catheter insertion site or the like. The dressing is preferably in a non-fluid state such that it can be handled manually and conveniently, and placed on a wound easily. Such a dressing may be referred to a “hydrogel dressing”. By hydrogel dressing, it means the dressing is made of a gel-like substance having a relatively high water content.

Preferably, the dressing may comprise at least 80 wt % of purified water. With such a relatively high water content, the dressing is fairly soft, wounds which are often very sensitive are often less irritated thereby.

Advantageously, the infection control agent may be an anti-bacterial agent for growth of bacteria. The infection control agent may also be an anti-inhibiting microbial agent for inhibiting growth of microbial. Further, the infection control agent may comprise a chlorhexidine composition selected from the group including hibitane, chlorhexidine acetate, chlorhexidine gluconate and chlorhexidine hydrochloride. These compositions are useful in inhibiting growth of microbes and yet they have relatively low cytotoxic effect on wounds treated or the surrounding thereof. It is to be noted that these compositions do not interfere with cross-skin linking of the polymers or monomers during formation of the dressing.

Suitably, the dressing may comprise 0.025 to 0.75 wt % of the infection control agent. The content of the infection control agent may often be important because if the content were too high the formation of the cross-linked structure might be prevented. Even if the structure could be formed, the high content of the infection control agent would cause irritation to the wound treated. If the infection control agent content were too low, then it would not have sufficient strength to control infection and the wound treated may still be infected. This is particular so since the dressing may have a relatively high water content which could foster growth of microbes such as bacteria and fungus.

In particular, the cross-linked structure may comprise a synthetic polymer such as polyacrylamide, polyvinylpyrolidone, their monomers or their mixtures, or a natural polymer such as gelatine or agar, or mixtures thereof.

The dressing may further comprise a plasticizing agent, and the agent may be selected from the group including ethylene glycol, polyethylene glycol, propylene glycol and polypropylene glycol. This agent serves to enhance the flexibility, adhesiveness and/or permeability of the dressing.

Preferably, the sheet material may be a moisture balancer, a disinfectant or an antiseptic agent for the wound, the surgical incision, the catheter insertion site or the like. Moisture and disinfections often are needed by wounds in their healing.

Advantageously, the cross-linked structure may comprise means for absorbing discharges such as pus or exudates from the wound, the surgical incision, the catheter insertion site or the like. The cross-linked structure may also release the infection control agent and moisture to the wound and disinfect the wound in exchange for the discharges. The agent can also kill the microbes in the absorbed exudates.

According to a second aspect of the present invention, there is provided a method of dressing a wound, a surgical incision, a catheter insertion site or the like, comprising applying a dressing as described above.

According to a third aspect of the present invention, there is provided a method of manufacture of a dressing for a wound, a surgical incision, a catheter insertion site or the like, comprising the steps of: (a) preparing a mixture of ingredients including at least 80 wt % of purified water, 0.025 to 0.75 wt % of an agent for controlling infection or inhibiting growth of bacteria or microbes, and monomers and/or polymers to be cross-linked together, (b) placing said mixture in a mould having a desired shape, and (c) solidifying said mixture by subjecting said mixture in said mould to an ionizing radiation (e.g. gamma radiation) in the magnitude of at least 20 kGy or an electron beam and cross-linking the monomers and said polymers in said mixture to form a crosslinked polymer sheet structure. Experiments have shown that excessive use of an infection control agent would prevent the formation of the polymerized structure.

Preferably, the agent may comprise a chlorhexidine composition selected from the group including hibitane, chlorhexdine acetate, chlorhexdine gluconate and chlorhexidine hydrochloride. These compositions are particularly useful in that while they are effective with inhibiting growth of microbes they do not interfere with the cross-linking of the monomers and/or polymers in the mixture.

Advantageously, the monomers or the polymers are natural polymers, synthetic monomers or synthetic polymers. When the synthetic monomers or polymers are used the mixture may comprise 5 to 12 wt % thereof; and when the natural polymers are used the mixture may comprise no more than 4 wt % thereof. Further, the monomers may be are selected from acrylamide, vinylpyrolidone. The natural polymer may be selected fromgelatine and agar.

Suitably, the ionizing radiation may be gamma radiation; the strength of the radiation may be in the range of 25 to 40 kGy.

According to a fourth aspect of the present invention, there is provided a method of manufacture of a dressing for a wound, a surgical incision, a catheter insertion site or the like, comprising the steps of (a) preparing a mixture of ingredients including at least 80 wt % of purified water, 0.025 to 0.75 wt % of a chlorhexidine composition for controlling infection or inhibiting growth of bacteria or microbes, and monomers and/or polymers to be cross-linked together, (b) placing the mixture in a mould having a desired shape, (c) solidifying said mixture by subjecting said mixture in the mould to an ionizing radiation such as gamma radiation or electron beam. Preferably, the ionizing radiation may be in the magnitude of at least 20 kGy. The ionizing radiation may be gamma radiation although any suitable radiation that could induce the cross-linking would also be appropriate.

### DETAILED DESCRIPTION OF THE PRESENT INVENTION AND PREFERRED EMBODIMENTS

One aspect of the present invention relates to a hydrogel dressing which is in a non-fluid state or a gel-like substance. It has effects in not only covering a wound treated therewith but also releasing an appropriate quantity of an infection controlling agent to the wound and balancing the moisture required by the wound. It can also absorb exudates from the wound and kill the microbes in the absorbed exudates. The following illustrates several embodiments of hydrogel dressing made in accordance with the present invention.

### EXAMPLE I

A solution was prepared by mixing the following ingredients together.

| Ingredients | Content (part by weight) |
|---|---|
| chlorhexidine gluconate | 0.5 |
| vinylpyrolidone and/or poly | 11 |
| vinylpyrolidone | |
| ethylene glycol, polyethylene glycol | 2 |
| Agar | 1.5 |
| purified water | 85 |
| Total | 100 |

The above ingredients were mixed together in a clean environment and a mixture in the form of a solution was produced. The solution was then poured into a Petri dish of suitable size such that the solution formed a liquid layer with a thickness of about 4 mm. The Petri dish was then tightly covered with a polyethylene film and then heat sealed around the edges thereof. The Petri dish together with the solution therein was exposed to a gamma radiation of 30 kGy. The solution then became solidified forming a non-fluid sheet after the crosslinking of vinyl pyrolidone, polyvinyl pyrolidine and agar.

Chlorhexidine gluconate is used as an infection-controlling agent which inhibits growth of microbes. Vinyl-pyrolidone or polyvinyl-pyrolidone is a synthetic monomer/polymer which is cross-linkable to form a cross-linked matrix structure. Ethylene glycol/polyethylene glycol is used as a plasticizing agent to enhance the physical properties of the dressing. Agar is a natural polymer and is also cross-linkable to form a or another cross-linked structure, and form a gel-like substance. When the solution as in the example is exposed to the radiation, the solution becomes solidified and a sheet-like dressing is formed.

A dressing made by the above method is sterile, elastic, disinfecting and transparent. Due to the disinfecting nature of the dressing, no additional disinfectant may be applied to the wound. Due to its transparent characteristics, the healing of the wound can be observed without frequent removal of the dressing. Due to the cross-linked matrix structure of the dressing, it is able to absorb exudates from wound treated therewith and minimize the change of dressings. Due to the relatively high water content and elastic nature of the dressing, it is gentle to the wound and can balance the moisture of the wound. Due to the presence of the infection-controlling agent, infection of the wound becomes very unlikely. It is also to be noted that unlike conventional dressings which become no longer sterile once they are exposed to the surroundings, a hydrogel dressing made in accordance with the present invention remains sterile even after it is exposed to the surroundings. The sheet structure of the dressing conforms to the wound very well and provides a barrier to bacteria from the surroundings. The dressing with these characteristics together is desirable for assisting wound healing.

### EXAMPLE II

A solution was prepared by mixing the following ingredients together.

| Ingredients | Content (part by weight) |
|---|---|
| chlorhexidine hydrochloride | 0.05 |
| vinylpyrolidone and polyvinylpyrolidone | 6 |
| ethylene glycol and polyethylene glycol | 1.5 |
| Agar | 1.5 |
| Purified | 90.95 |
| Total | 100 |

Similar to Example I, the above ingredients were mixed together in a clean environment and the solution is formed. The solution was then processed following the same steps as in Example I, except a gamma radiation with a strength of 35 kGy was used.

A dressing formed from the above method exhibits properties similar to the dressing obtained from Example 1.

### Examples III and IV

The above two examples were repeated but during the radiation step, the solution was exposed to an electron beam instead of gamma radiation. The resultant dressings were also found to possess similar characteristics and effectiveness.

Experiment I

A number of experiments were conducted in order to determine the effectiveness of the dressing in inhibiting bacteria growth by a method called zone of inhibition method (ZOI). In one experiment (Experiment I), segments of the dressing from Example I were removed. The segments were firstly cut into a size of about 1 cm×1 cm. They were then immersed and soaked in about 1 ml of water for 10 minutes. The segments of the dressing were then removed from the water and excess water thereof was drained away and absorbed on a sterile agar surface. The segments were then laid on a number of agar plates with each of the agar plates plated with a particular bacteria. Please see Table 1 below for the different types of bacteria tested on. The agar plates were then incubated for 24 hours.

After incubation, the agar plates were observed as to the growth of the bacteria. Measurements were taken to see how far away from the segments there was bacteria growth. Table 1 below summaries the results. For example, when tested on an agar plate with Methicillin-Resistant Staphylococcus aureus, there were no bacteria growth beneath the segment and within the boundary 5.75 mm away from the segment. In other words, it is shown that the dressing is effective in inhibiting growth of this bacteria.

TABLE I

| Microorganism | Sample | |
|---|---|---|
| Pseudomonas aeruginosa | >3.5 | mm |
| Staphylococcus aureus | >5.5 | mm |
| Acinetobacter baumannii | 2.5 | mm |
| Acinetobacter baumannii 4454 | >2 | mm |
| Methicillin-Resistant Staphylococcus aureus | 5.75 | mm |
| Enterococcus faecalis | >4.5 | mm |
| Escherichia Coli | 4 | mm |
| Proteus vulgaris | >1.5 | mm |
| Entrobacter cloacae | 3 | mm |

Experiments have shown that the above described hydrogel dressings may not only be used for covering wounds but also disinfecting the wounds and minimizing the risks associated with infection.

1. A hydrogel dressing for covering or treating a wound, a surgical incision, a catheter insertion site or the like, comprising a non-fluid sheet material including a structure of cross-linked polymers and/or monomers having suspended therein an agent for controlling infection of the wound, the incision, the catheter insertion site or the like.

2. A dressing as claimed in claim 1 comprising at least 80 wt % of purified water.

3. A dressing as claimed in claim 1 wherein said infection control agent is an anti-bacterial agent.

4. A dressing as claimed in claim 1 wherein said infection control agent is an anti-microbial agent.

5. A dressing as claimed in claim 1 wherein said infection control agent comprises a chlorhexidine composition selected from the group including hibitane, chlorhexidine acetate, chlorhexidine gluconate and chlorhexidine hydrochloride.

6. A dressing as claimed in claim 1 comprising 0.025 to 0.75 wt % of said infection control agent.

7. A dressing as claimed in claim 1 wherein said cross-linked structure comprises a synthetic polymer such as polyacrylamide, polyvinylpyrolidone, their monomers or their mixtures, and/or a natural polymer such as gelatine or agar, or mixtures thereof.

8. A dressing as claimed in claim 1 further comprising a plasticizing agent.

9. A dressing as claimed in claim 8 wherein said plasticizing agent is selected from the group including ethylene glycol, polyethylene glycol, propylene glycol and polypropylene glycol.

10. A dressing as claimed in claim 1 wherein said sheet material is a moisture balancer for the wound, the surgical incision, the catheter insertion site or the like treated.

11. A dressing as claimed in claim 1 wherein said sheet material acts as a disinfectant or an antiseptic agent.

12. A dressing as claimed in claim 1 wherein said cross-linked structure comprises means for absorbing discharges such as pus or exudates from the wound, the surgical incision, the catheter insertion site or the like.

13. A method of dressing a wound, a surgical incision, a catheter insertion site or the like, comprising applying thereon a dressing as claimed in claim 1.

14. A method of manufacture of a dressing for a wound, a surgical incision, a catheter insertion site or the like, comprising the steps of:
(a) preparing a mixture of ingredients including at least 80 wt % of purified water, 0.025 to 0.75 wt % of an agent for controlling infection or inhibiting growth of bacteria or microbes, and monomers and/or polymers to be cross-linked together;
(b) placing said mixture in a mould having a desired shape; and
(c) solidifying said mixture by subjecting said mixture in said mould to an ionizing radiation in the magnitude of at least 20 kGy or an electron beam and cross-linking said monomers and said polymers in said mixture to form a crosslinked polymer sheet structure.

15. A method as claimed in claim 14 wherein said agent comprises a chlorhexidine composition selected from the group including hibitane, chlorhexdine acetate, chlorhexdine gluconate and chlorhexidine hydrochloride.

16. A method as claimed in claim 14 wherein said monomers or said polymers are natural polymers, synthetic monomers or synthetic polymers.

17. A method as claimed in claim 16 wherein said mixture comprises 5 wt % to 12 wt % of said synthetic monomers or polymers.

18. A method as claimed in claim 16 wherein said mixture comprises no more than 4 wt % of said natural polymers.

19. A method as claimed in claim 14 wherein said polymers are selected from the group including polyacrylamide, polyvinylpyrolidone, gelatine and agar.

20. A method as claimed in claim 14 wherein said ionizing radiation is gamma radiation.

21. A method of manufacture of a dressing for a wound, a surgical incision, a catheter insertion site or the like, comprising the steps of:
(a) preparing a mixture of ingredients including at least 80 wt % of purified water, 0.025 to 0.75 wt % of a chlorhexidine composition for controlling infection or inhibiting growth of bacteria or microbes, and monomers and/or polymers to be cross-linked together;
(b) placing said mixture in a mould having a desired shape; and
(c) solidifying said mixture by subjecting said mixture in said mould to an ionizing radiation.

22. A method as claimed in claim 21 wherein said ionizing radiation is in the magnitude of at least 20 kGy.

23. A method as claimed in claim 21 wherein said ionizing radiation is gamma radiation or an electron beam.
